(19) United States
(12) Patent Application Publication
(10) Pub. No.: US 2005/0021088 A1
(43) Pub. Date: Jan. 27, 2005

(54) Systems containing temperature regulated medical devices, and methods related thereto

(76) Inventors: Peter S. Schuler, Westwood, MA (US); Douglas U. Gwost, Shoreview, MN (US); \Wolfgang Daum, Groton, MA (US); Robert Schwartz, Minneapolis, MN (US)

Correspondence Address: Pepper Hamilton, 50th Floor, One Mellon Center 500 Grant Street, Pittsburgh, PA, 15219, US

(21) Appl. No.: 10/493,874

(22) PCT Filed: Oct. 29, 2002
(86) PCT No.: PCT/US02/34587

Related U.S. Application Data
(60) Provisional application No. 60/352,141, filed on Oct. 29, 2001.
(60) Provisional application No. 60/395,784, filed on Jul. 15, 2002.

(51) Int. Cl. A61N 1/00; H05B 11/00

(52) U.S. Cl. 607/1; 219/212

(57) ABSTRACT

Disclosed are systems for the application of heat to an area of the body of a mammal, a system including a device fabricated from or coated with a material comprising of a non-metal matrix and susceptor particles, a non-invasive inductor and magnetic circuit for heating the particles by transmitting an alternating magnetic field (AMF), and an alternating current generator that provides an alternating current to the inductor. Also disclosed are methods related to the non-invasive application of heat to mammalian tissue. These systems and methods are useful where heat must be applied in a controlled manner to avoid undesired damage to tissue.

### CROSS REFERENCE TO RELATED APPLICATIONS

This is a non-provisional application claiming the benefit of and priority to provisional patent application No. 60/352,141 filed on Oct. 29, 2001, and provisional patent application No. 60/395,784, filed on Jul. 15, 2002.

### FIELD OF THE INVENTION

The present invention relates to systems containing temperature regulated devices that utilize alternating magnetic frequency (AMF). More specifically, the present invention relates to systems containing medical devices, such as probes and implants, heated by an AMF source, which are used for various medical treatments and procedures in the treatment of humans and animals. The devices of these systems are capable of being repeatedly and controllably heated using materials possessing a Curie temperature.

### BACKGROUND

Heat has various effects on human or animal tissue. At lower temperatures, the growth of the cells is halted. Raising the temperature causes programmed cell death (apoptosis), in which cells and their nuclei shrink, condense and break apart to be ultimately phagocytized by other cells. Raising the temperature even higher results in cells swelling, bursting and dying immediately (necrosis) or the tissue being destroyed (ablation).

There are many areas in the body in which heat can be used therapeutically, for example, treatment of vascular and cardiovascular plaque, treatment of cancerous lesions, or the removal of moles of the skin. Heat can also be used to heat auxiliary medical devices.

1. Treatment of Vascular and Cardio-Vascular Plague

There are a number of disease states that are treatable by applying a focused and controlled heat source to destroy blockages or growths in body parts using ablation. For example, a long known problem in patients suffering from heart disease is the blockage of coronary or other arteries due to the build up of calcified or hard plaque. If the plaque is not removed, the diameter of the artery decreases, restricting the flow of blood. Atherectomy is a procedure for opening the coronary arteries blocked by plaque. Angioplasty, laser angioplasty and rotating shavers are typical procedures for opening of arteries blocked by plaque. These techniques bear the danger of damaging the vessel wall.

There are devices for use in cardiovascular medicine (e.g., U.S. Pat. No. 5,087,256 to Taylor). An example of such a device is a thermal atherectomy catheter, for use in blood vessels and the like, that comprises a tip of high magnetic permeability including a cylindrical body terminating at one end in an enlarged head and at its other end in an enlarged collar, a coil of wire adapted to be connected to a source of current wound about said cylindrical body essentially abutting said head and removed from said collar. Thus, the coil that generates the magnetic field used to heat the tip is located on the tip and, hence, is placed inside the target to be treated. Such a device presents a limitation that the probe must be connected to wires that connect to the external power source.

Some plaques are “hard and solid” (calcified plaque), and the others are “soft and squishy”. This soft plaque has been called “vulnerable plaque” because of its tendency to burst or rupture. Vulnerable plaques are usually those causing only mild to moderate stenosis and having a lipid-rich core and a thin, macrophage-dense, collagen-poor fibrous cap. Factors affecting plaque rupture include mechanical injury, circadian rhythm, inflammation, and infection. Progressive thrombosis and vasospasm may follow plaque rupture. Present methods of treatment using heat (e.g., U.S. Pat. No. 5,906,636 to Casscells) include treating inflamed regions containing deleterious immune cells with temperatures in the range of 38.5° C. to 44.0° C. for about 5 minutes to 60 minutes.

Due to the successes demonstrated with these methods, devices that utilize heat for inducing tissue or cell necrosis, or programmed cell death (apoptosis), would be useful in other intravenous applications, vascular applications, urinary treatments, e.g., urethral or gall bladder, applications.

2. Treatment of Cancerous Lesions

Heat can also be applied to treat cancerous lesions (e.g. U.S. Pat. No. 5,133,710 to Carter and U.S. Pat. No. 6,007,474 to Rydell). Present devices for such treatments include a heater system for subjecting body tissue to hyperthermia or higher temperatures comprising a heater including a core of a material having high magnetic permeability (μ) and low electrical conductivity. The core has an elongated dimension and is completely covered with a sheath of electrically conductive material which has a permeability of more than an order of magnitude less than the permeability of the core. The system also contains a coil structure for producing an AMF, along with a means for locating the coil structure relative to the heater to induce a current therein. In this system, the AMF source causes the ferromagnetic material to generate a secondary field that causes the conductive sheath to heat through eddy current, without temperature control. When the dielectric material heats, the heat is transferred to the ferromagnetic material. At the Curie temperature of the ferromagnetic materials, the core stops generating the secondary field and stops the sheath from heating. However, there may be a lag time in heating and secondary effects, resulting in less accurate temperature control.

Another device involves the induction heating of implanted metallic rods to heat prostate tissue to the Curie temperature of the metallic rods. However, pure metallic devices should not be used for a variety of medical and technical reasons, such as toxicity, biocompatibility, bioabsorbability or stiffness. Thus, there is a need for a greater variety of materials usable for inductive heating in medical device technology.

3. Non-invasive Inductive Heating of Auxiliary Medical Devices

Metallic implanted medical devices can be heated inductively using appropriate technology (e.g., U.S. Pat. No. 6,238,421 and EP 1,036,574). However, such technology has limitations, for example, it can only be used with certain metallic implants which heat uncontrollably when exposed to AMF. Control mechanisms require implantable temperature probes to determine when a maximum temperature has been achieved. The operator either manually or automatically can reduce the power to the AMF generator in an attempt to control the temperature.

4. Other Applications of Implants or Probes

There are numerous other medical therapies in which cells and tissue are modified by temperature (e.g., U.S. Pat. No. 6,261,311 and U.S. Pat. No. 5,624,439). Examples include the use of elevated temperatures of up to 60° C. to 90° C. to cause shrinkage of injuring spinal disks, and the reduction of snoring by heat caused shrinkage of enlarged turbinates.

Heat can also be applied to the skin (dermal and subdermal) for therapeutic and cosmetic purposes, such as removal of cancerous lesions, moles and age spots, coagulation of intraluminal spider veins, and reduction of wrinkles.

Current heating devices tend to heat uncontrollably and this overheating is prevented largely by the experience and skill of the practitioner/operator.

### SUMMARY OF THE INVENTION

Examples of therapeutic uses of heat include treatment of vascular and cardio-vascular plaque, treatment of cancerous lesions, and the removal of moles of the skin. However, a major problem with heating devices for such applications has been the inability to control the rate and temperature of heating, resulting in undesired damage to tissue.

In view of the above, there is a need for a medical device that can effectively heat parts of the body to a predetermined temperature without damaging any tissue. Such a device may contain a probe that can be directly inserted into a body part to heat a particular area or tumor repeatedly. It is preferable that such a probe not have any external wires or metal components. It is also preferable to have a heating probe that heats up to a predetermined and controlled temperature to prevent burning and/or causing other tissue damage. It is also desirable to have methods for heating tissue in a safe and effective manner. The probe may be heated non-invasively.

It is, therefore, an object of the present invention to provide a system for the therapeutic application of heat to an area of the body of a mammal. The heating may be non-invasive.

It is another object of the present invention to provide implantable medical devices manufactured from non-metallic materials that can be imbedded or coated with Curie temperature materials to control the maximum temperature.

It is yet another object of the present invention to provide a medical device that that can be implanted in the body for at least one hour and that can be repeatedly heated.

It is a further object of the present invention to provide methods of applying heat to a mammalian body that involves the application of AMF to a device that is applied to mammalian tissue. The application of heat may be performed non-invasively.

The present invention pertains to a system for applying heat to an area of the body of a mammal, the system including a device fabricated from or coated with a material comprising a non-metal matrix and susceptor particles, an inductor and magnetic circuit for heating the particles by transmitting an alternating magnetic field (AMF), and an alternating current generator that provides an alternating current to the inductor. The present invention also pertains to a device that is a part of the heat application system and that can be implanted in the body. The matrix of such a device can be a plastic, a thermoset, a thermoplastic, an elastomer, a ceramic, or a gel. The susceptor particles of such a device may have a Curie temperature.

The present invention further pertains to methods related to the application of heat to tissue. One such method includes the application of heat to a mammalian body that includes applying to a mammal tissue a device that is partially or completely fabricated from or coated with a non-metal matrix containing susceptor particles, and applying an AMF to the device. The methods of the present invention provide for the application of heat to mammalian tissue in a safe and effective manner, with controlled and repeatable heating.

The above summary of the present invention is not intended to describe each illustrated embodiment or every implementation of the present invention. The figures and the detailed description which follow more particularly exemplify these embodiments.

### BRIEF DESCRIPTION OF THE FIGURES

The invention may be more completely understood in consideration of the following detailed description of various embodiments of the invention in connection with the accompanying drawings, in which:

FIG. 1 schematically illustrates a medical device according to an embodiment of the present invention; and

FIG. 2 presents a graph showing the relationship between magnetization and temperature, for a material that has a Curie temperature, T_{c}.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF EXEMPLARY EMBODIMENTS

1. System for Application of Heat to Mammalian Body

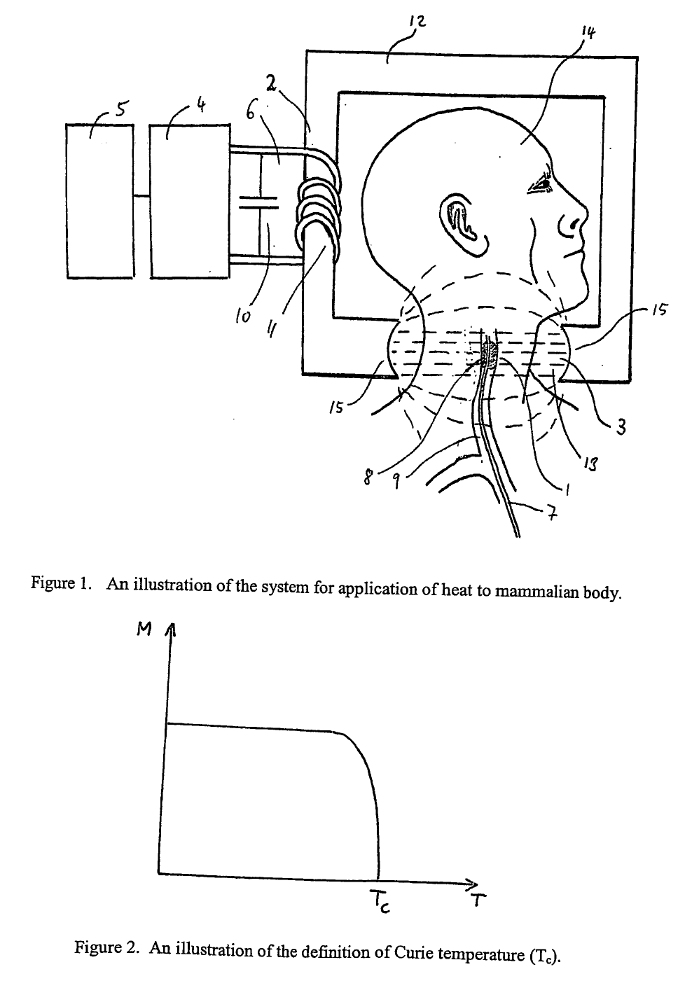

As illustrated in FIG. 1, one particular embodiment of a system for the application of heat to mammalian body or body part comprises a device 1, fabricated from a material comprising a non-metal matrix and susceptor particles embedded therein, an inductor 2 for heating the particles by transmitting an alternating magnetic field (AMF) 3, and an alternating current power source 5 with a resonant circuit (or impedance matching network) 4 for providing the alternating current to the inductor 2. The AMF 3 generated by the inductor 2 is directed at the device 1 by a magnetic circuit 12. The alternating current power source 5 and the resonant circuit (or matching network) 4 are collectively called the alternating current generator 6.

In this particular embodiment, the device 1 is a carotid catheter comprising a catheter tube 7 and an expandable balloon 8, similar to a percutaneous transluminal coronary angioplasty (PTCA) balloon, which is inserted into a carotid artery 9. The balloon 8, or a balloon with expanding media, or a coating on the balloon 8, or any combination of thereof, may be fabricated from a non-metal matrix embedded with susceptor particles.

The matrix material of the balloon 8 in this embodiment may be an elastomeric material. The embedded susceptor particles can be of any composition described herein that are susceptible to AMF, meaning that they absorb the AMF energy and transform the energy into heat to cause the temperature of the particles to rise. In this particular embodiment, catheter tube 7 does not comprise susceptor particles, hence only a portion of the device 1 is fabricated from a non-metal matrix in which susceptor particles are embedded.

The alternating current power source 5 may be an RF oscillator or RF amplifier. If the alternating current power source 5 is an RF oscillator, then 4 is a resonant circuit. Alternatively, if the alternating current power source 5 is an amplifier, then 4 is an impedance matching network.

In this embodiment, the AMF 3 produced by the inductor 2 is coupled into the magnetic circuit 12 having a gap 13 into which the patient or a body part of the patient 14 is located. The magnetic circuit 12 is constructed from a magnetic material, such as any type of ferrite, capable of guiding the magnetic flux. Alternatively, the patient may be placed within the inductor 2 itself, or the inductor may be invasively placed within the patient, to heat the medical device.

It will be appreciated that the poles 15 of the magnetic circuit 12 may be shaped to produce a desired magnetic filed profile. In the illustrated embodiment, the poles 15 are provided with concave shapes to enhance the homogeneity of the alternating magnetic field 3 in the gap 13. Further, the poles 15 may be formed from pole pieces to create an adjustable gap, so as to permit any part of the body to be treated effectively.

Either immediately or anytime after the implantation of a device 1 in the patient 14, the device 1 can be repeatedly heated with the AMF 3 energy. When the AMF 3 is applied to the device 1, the susceptor particles heat up. If the susceptor particles possess a Curie temperature, they only heat up to the Curie temperature. The Curie temperature (T_{c}) is defined as the temperature at which a material's magnetic property undergoes a transition from ferro- or ferrimagnetic to paramagnetic.

The medical balloon device illustrated in FIG. 1 is in contact with the vessel wall of the carotid artery and may be heated up to 43° C. The heat of the expanded and heated balloon flows into the vessel wall to cause apoptosis in the fatty macrophages of the vulnerable plaque.

In FIG. 2, the magnetization M of a material is plotted versus temperature T. The M-T graph can differ from material to material. The temperature at which the magnetization approaches zero is referred to as the Curie temperature of the material. The Curie temperature of certain metals, metal alloys and metal oxides is used to limit the temperature of the device constructed from such materials to a defined maximum.

2. Susceptor Particles

The devices of the systems and related methods of the present invention utilize preferred particles, which are herein referred to as “susceptors” or “susceptor particles”. These susceptor particles can be embedded in a non-metal matrix material, such as a thermoplastic, a plastic, a thermoset, an elastomer, a ceramic, or a gel. The susceptors are selected to couple with the AMF such that they rapidly and controllably heat. If the susceptors possess a Curie temperature, they will heat to a desired maximum temperature (Curie temperature). Utilizing susceptor particles possessing a Curie temperature is advantageous in that there is a built-in thermostatic control whereby the degree of heating can be controlled in a precise manner. In the presence of an electromagnetic field, the susceptor particles in the matrix heat rapidly to the predetermined Curie temperature. This built-in thermostatic control offers a way to prevent undesirable overheating.

The mechanism of heating can be, but is not limited to, hysteresis heating, Néel heating, Brownian heating, eddy current heating, dielectric heating, or any combination of these. For example, with electrically conductive magnetic materials, heating can occur by both eddy current and hysteresis losses. In typical non-conducting magnetic materials, heating primarily occurs by hysteresis losses. This mechanism exists as long as the temperature is maintained below the Curie temperature (T_{c}) of the material. At the Curie point, the originally magnetic material becomes essentially non-magnetic.

The devices of the systems of the present invention enable the tailoring of the temperature of the probes via the selection of an appropriate susceptor(s) based upon the desired application. Susceptors useful herein can be any that are known in the art. Preferred susceptors for use in the present invention possess a Curie temperature.

Examples of preferred susceptors include metal oxide compounds that have the following general structures: SrFe₁₂O₁₉, Meₐ-2W, Meₐ-2Y, and Meₐ-2Z, wherein 2W is BaO:2MeₐO:8Fe₂O₃, 2Y is 2(BaO:MeₐO:3Fe₂O₃), and 2Z is 3BaO:2MeₐO:12Fe₂O₃, and wherein Meₐ is a divalent cation. The divalent cation is preferably selected from Mg, Co, Mn and Zn. Other examples are 1 Me_{b}O: 1 Fe₂O₃, where Me_{b}O is a transition metal oxide selected from Ni, Co, Mn, and Zn

Further examples of susceptors are metal alloys: La_{0.8}Sr_{0.2}MnO₃; Y₃Fe₅₋ₓMₓO₁₂ where M is Al, or Gd and 0<x<2; metal alloys of any combination of Pd, Co, Ni, Fe, Cu, Al, and Si; metal alloys of any combination of Gd, Th, Dy, Ho, Er, and Tm with any combination of Ni, Co, and Fe; and metal alloys RMn₂X where R is a rare earth, such as La, Ce, Pr, or Nb and X is either Ge or Si.

Examples of more preferred susceptors include:

| Susceptor | Curie temperature |
|---|---|
| Ni 28% Cu | 60° C. |
| Ni 29.6% Cu | 50° C., at 90 kHz |
| Ni 29.6% Cu | 60° C., at 100 kHz |
| NiPd | 43° C.-58° C. |
| Pd 6.15% Co | 50° C. |
| Ni 4% Si | 50° C. |
| (Ni,ZnO)Fe₂O₃ | 80° C., at 80 micrometers |
| La_{0.8}Sr_{0.2}MnOₓ | 48° C. |
| Y₃Fe₅—_{X}Al_{X}O₁₂ | 22° C.-140° C. where x = 1.7-1.0 |

The susceptor particles useful in the present invention can be of any size. In general, the particles are from about 10 nanometers to about 500 microns in the longest dimension. In certain embodiments, the preferred particles are less than 1 micron in the longest dimension. More preferred particles range in size from about 20 nanometers to about 200 nanometers in the longest dimension. In certain other embodiments, the particle size ranges from about 1 micrometer to about 50 micrometers in the longest dimension.

The Curie temperature of the susceptors useful herein ranges from about 35° C. to about 150° C., depending on the application. For some preferred embodiments, the Curie temperature is in the range of from about 37° C. to about 75° C. In certain other embodiments, the preferred Curie temperature is in the range from about 38° C. to about 45° C.

Susceptor particles that are not biocompatible with mammalian tissue may be coated with a bioinert or biocompatible coating. A particle may be coated with pure titanium, a titanium alloy, or a biocompatible polymer to permit the permanent implantation of the device is the body. This implanted device can then be heated as deemed necessary. The coating may be a metallic material to enhance the eddy current effect, if needed. In other embodiments, it may be desirable to have a non-conducting coating, such as Teflon or another plastic material. The coating may also serve as a good heat conductor. There are coatings, such as polyethylene, polylactic acid, polyethyleneglycol, polyalkylcyanoacrylate, albumin or dextran, that can increase the ability of the susceptor particle to be resorbed by the body metabolism, if mixed into a resorbable gel. For resorbable gels, the particle size is important; the smaller the particle, the greater its resorbtion. Hence, particles used in devices intended to remain as a permanent implant may be of larger size.

A matrix material may be embedded with one or more types of susceptor particles. In certain embodiments, the matrix may comprise various types of susceptor particles that possess the same or different Curie temperature, or even some particles without a Curie temperature. In other embodiments, it may be desirable to have different size particles. Certain other devices comprise one or more portions with matrixes that contain different types of particles, particles with different densities or even portions in which the density of the particles form a gradient from one side to the other.

The density of the particles distributed in the matrix-particle mixture yield the composition density, or herein referred to as “density”, is the sum of the volumes of the individual particles divided by the volume of the matrix-particle mixture and multiplied by 100 to arrive at the percentage value. Depending on the desired maximum temperature, matrix-particle mixture densities may be higher or lower, and can vary from about 5% to about 95%, by volume. Densities between 35% and 75%, by volume, are preferred for use in the present invention.

3. Matrix Materials

The matrix is a non-metal material, preferably a plastic, a thermoplastic, an elastomer, a ceramic, or a gel. Preferred plastics include any type of plastic known in the art that is biocompatible, moldable, has good chemical resistance, and, has a melting temperature higher than the Curie temperature of the imbedded susceptors if the susceptor possesses a Curie temperature. In preferred embodiments, the matrix material is a thermoplastic that comprises poly(etheretherketone) (PEEK), polyetherketoneketone (PEKK), poly(etherimide) (PEI), polyphenylene sulfide (PPS), poly(sulfone) (PSU), polyethylene terephthalate (PET), polyester, polyamide (PA), polypropylene (PP), polyurethane (PU), polyphenylene oxide (PPO), polycarbonate (PC), PP/MXD6 (MXD6 is a Mitsubishi trademark for a type of polyamide or nylon), polyethylene (PE), or any combination thereof. Examples of preferred plastic materials include teflons and nylons. Elastomers useful herein include silicon, latex or any other artificial or natural rubber.

Examples of ceramics useful herein include rigid silicon carbides, and flexible ceramic materials that have elastic properties similar to metals and metallic materials. An example of this is described in “A High-Strain-Rate Superplastic Cerarmic” by B.-N. KIM et al., Nature 413, 288-291 (2001), incorporated herein by reference.

Gels useful in the present invention may be of a natural source, such as starch, or an artificial source, such as polyacrylamide. The gel material may also be a sugar based substance, such as glactose, a wax based substance, such as esters of long-chain carboxylic acids with long-chain alcohols, a fat based substance, such as triesters of glycerol with three long-chain carboxylic acids, or a silicone based substance. The gel material may be a hydrogel in its hydrated or dehydrated form. The gel material may also be selected from the group consisting of acrylonitriles, acrylic acids, polyacrylimides, acrylimides, acrylimidines, polyacrylonitriles, and polyvinylalcohols. The gel material may be derived from petrochemical oils or natural oils such as coconut, corn, olive or bean oils.

The matrix material may be an absorbable or bioabsorbable material.

4. AMF Source

Many different types of fundamental waveforms of AMF may be useful in the present invention. Examples of waveforms useful herein include sinusoidal, triangular, square, sawtooth, and trapezoidal current waveforms. The amplitude of the waveform may be modulated. The shape of the amplitude modulation envelope may typically be sinusoidal, square, triangular, trapezoidal, sawtooth, any variation or combination thereof, or may be some other shape.

The AMF 3 produced by generator 6 may be constant or pulsed. Pulse width is traditionally defined as the time between the −3dBc points of the output of a square law crystal detector. Because this measurement technique is cumbersome in this application, we use an alternate definition of pulse width. For the purpose of this invention, pulse width may be defined as the time interval between the 50% amplitude point of the pulse envelope leading edge and the 50% amplitude point of the pulse envelope trailing edge.

The pulse width may also be modulated.

The pulse repetition frequency (PRF) is defined as the number of times per second that the amplitude modulation envelope is repeated. The PRF typically lies between 0.0017 Hz and 1 MHz. The PRF may also be modulated. The duty cycle may be defined as the product of the pulse width and the PRF, and is thus dimensionless. In order to be characterized as pulsed, the duty of the alternating current generator must be less than unity (or 100%).

The AMF 3 may be constrained to prevent heating healthy tissue to lethal temperatures (typically ≧43° C.). This may be accomplished in a variety of ways:
The peak amplitude of the AMF may be adjusted. The PRF may be adjusted.
The pulse width may be adjusted.
The fundamental frequency may be adjusted.

These four characteristics may be adjusted to maximize the heating rate of the particles and, simultaneously, to minimize the heating rate of the healthy tissue located within the treatment volume. These conditions may vary depending upon tissue types to be treated, thus the operator may determine efficacious operation levels. In one embodiment, one or more of these characteristics may be adjusted during treatment based upon one or more continuously monitored physical characteristics of tissue in the treatment volume by an interventionally located temperature probe, which might be glass fiber based. This information may then be supplied as input to the generator or the operator to control the generator.

The generator output may be adjusted so that the peak AMF strength is between about 10 and about 10,000 Oersteds (Oe). Preferably, the peak AMF strength is between about 20 and about 3000 Oe, and more preferably, between about 100 and about 2000 Oe.

Additionally, the pulse width and/or the duty cycle may be adjusted to prevent heating healthy tissue. At typical pulse widths and duty cycles, eddy current heating is directly related to duty cycle. The capability to pulse the generator output and vary the duty cycle allows the benefits of operating at higher AMF amplitudes while maintaining a constant reduced tissue heating by reducing the duty cycle.

Although the desired frequency range is preferably between about 50 Hz and about 55 MHz, and more preferably between about 20 kHz and about 1 MHz, most preferably between about 50 kHz and about 500 kHz, the fundamental frequency may be adjusted to increase or decrease the rate of tissue heating as compared to the rate of hysteretic heating of the susceptors. Because the rate of hysteretic heating is directly related to frequency, and the rate of tissue heating is directly related to the square of the frequency, high AMF frequencies present a greater risk of damage to healthy tissue.

The devices of the systems of the present invention are preferably operated in a frequency range of from about 50 kHz to about 500 kHz. This lower range has no known detrimental effect on human tissue. However, the devices of the present invention can also be operated at higher frequencies if required by the particular application.

Typically, the rate of device heat-up is not of major concern, as it is with RF ablation devices. However, for devices that do not heat above 45° C., it is important for the device to heat up rapidly. For example, when treating vulnerable plaque with a heated balloon catheter, heat shock proteins (HSP) react to the heat. HSP are substances in the cells that protect the cell by deforming their shapes when heated. Such HSP have a defined reaction time. To be effective in the treatment of vulnerable plaque, the heating of the device must be quicker than this reaction time. The preferred heat-up time is under 10 seconds. Because the heat will have to flow from the susceptor particles into the matrix and from the matrix into the tissue, the particles have to heat even more rapidly.

5. Interventional Medical Devices

The devices of the systems of the present invention are different from prior known devices in that the inductor 2 that generates the AMF 3 is located outside the body and heats non-invasively. Thus, there are no wires, thermocouples, etc., attached to the device. One advantage of this feature is that the probe can be smaller, for example, than a conventional RF ablation device. Another advantage is that the therapy or procedure is less traumatic to the body. Consequently, certain embodiments of the device described herein may be permitted to remain within the body for repeated heating, e.g., for ongoing therapy or repetitive procedures.

The devices of the present invention can be formed entirely of non-metal matrix materials, e.g., plastic, ceramic, gel, embedded with susceptors, or can be coated with the matrix-susceptor combination. The maximum temperature of these devices is determined by the susceptor particles and the characteristics of the AMF 3 produced by the inductor 6 as defined herein.

The probe or other such devices can be used to cut, excise, soften or otherwise ablate, i.e., remove, human or animal body tissues, e.g., occlusions, tumors, biopsies, organs including skin, etc. Invasive examples include devices used in the removal of atherosclerotic plaque. Other embodiments include the use of these devices to reduce or eliminate total vascular occlusions or urinary obstructions, to treat and/or remove tumors, e.g., prostate or ovarian tumors. These devices can be used alone or in combination with other known treatments, e.g., PTCA (percutaneous transluminal coronary angioplasty). These devices are also useful for cauterization.

The devices of the present invention can be used for external treatment and therapy. Examples of external use include radial keratoplasty; mole, tattoo, or blemish removal; skin biopsies and various plastic surgeries. Thus, the devices of the present invention can be used to rapidly and accurately apply heat, for example, to a mole to remove it.

### EXAMPLES

### Example 1

A system to treat vulnerable plaque as illustrated in FIG. 1. Calcified plaque can also be treated in the same manner.

### Example 2

A system for tumor therapy. A probe, seed or capsule comprising a non-metal matrix and susceptor particles, is implanted in the tumor in the patient. The patient is exposed to non-invasive AMF and can optionally return for additional exposure to AMF as necessary to heat the probe as required by the necessary treatment. This technology can be used to remotely heat an implanted probe, seed or capsule that is coated or imbedded with an AMF susceptor to destroy or otherwise treat tumors or other masses (diathermy). One advantage of this approach is that after the probe or capsule is implanted, the heat therapy (thermotherapy) is repeated non-invasively.

This therapy can be used alone or in combination with other therapies. For example, a non-metal matrix and susceptor material can be attached to antibodies, polypeptides or other biologics to form a bioprobe that specifically attaches to the above-mentioned tumors or other masses. In this way, the AMF energy can be used to simultaneously heat both the susceptor on or in the probe or capsule and the bioprobe. The non-metal matrix and susceptor particles can also be adhered to or coated on a tumor or other tissue to heat and destroy said tumor or tissue. Such a device can also be used in any tissue accessible to minimal invasive devices, such as, but not limited to, BPH (benign prostatic hyperplasia), and tumors of the prostate, liver, brain, etc. Snoring can also be reduced by heat-induced shrinkage of enlarged turbinates. Spinal discs can also be reduced by heat-induced shrinkage.

### Example 3

Vulnerable plaques are usually those causing only mild to moderate stenosis and having a lipid-rich core and a thin, macrophage-dense, collagen-poor fibrous cap. A device fabricated from a gel matrix in which susceptors are embedded is injected into the regions of vulnerable plaque within the vessel wall in such a way that it remains therein for an extended period of time. Either immediately or anytime after the injection of the device, the device is heated repeatedly with AMF energy. If the gel matrix is heated to a preferred temperature between about 38.5° C. and about 44° C., apoptosis of the macrophage cells results, and whereby destroying vulnerable plaque.

### Example 4

Pharmaceuticals are incorporated into or coated onto the matrix in which the susceptors are also embedded to form a pouch or patch. The pouch or patch may be inserted in the body or attached to the skin. Either immediately or anytime after the implantation or attachment of the pouch or patch, the pouch or patch is heated repeatedly with AMF energy. The heating of the matrix causes the release of the pharmaceuticals into the body. These drug delivery devices may comprise bioresorbable or bioabsorbable matrix materials, and hence the devices will disappear over a period of time. The absorption rate of those devices may be enhanced by the heating of the device itself. The matrix for these devices might be a gel, a thermoplastic, or an elastomer.

Cardiovascular and other vascular stents tend to block or occlude after being put into use (instent-restenosis). Such stents can be fabricated from a non-metal matrix (plastics and ceramics) and susceptor particles or coated with the non-metal matrix embedded with susceptor particles as described herein. Either immediately or anytime after the implantation of the stent, the stent is heated repeatedly with AMF energy. Heat is generated on the surface of the stent, which is also known to prevent or reduce restenosis.

### Example 6

Shape memory alloy materials and devices may be exposed to a specific temperature in order to temporarily change the shape or geometry of the material, as dictated by the intended use. The shape memory alloy will return to its original shape or geometry when the alloy is cooled. Optionally, the heated shape or geometry of the shape memory alloy can be designed to prevent the shape memory alloy from returning to its original form by locking it into a new shape or geometry.

The shape memory alloy medical devices are well-suited for implantation in humans or animals. The susceptor material can either be coated on or imbedded into the device made from a shape memory alloy. Either immediately or anytime after the implantation of the device, the device is heated repeatedly with AMF energy to cause the shape or geometry of the device to change. The new shape or geometry is then locked in.

### Example 7

The inductor 2 is inserted into the human body to heat a medical device that has also been inserted into the body. The medical device is partly or wholly made out of a matrix comprising magnetic susceptible particles. For example, a vascular balloon catheter to treat plaque is positioned in a patient and the inductor 2 is introduced into the human body through a trocar and is located near the balloon of the catheter so that the susceptor particles in the balloon absorb sufficient AMF 3 to be heated to the desired temperature.

There are many other device configurations that one skilled in the art can design, examples of which include:
surgical tools, such as nails, screws, sutures, clips, filaments, fibers, trocars, open or minimal invasive surgical tools, internal and dermal ablation devices,
interventional tools, such as catheters, tubes, balloon catheters, balloons, balloon expanding media, guide wires, needles of any kind, localizers,
implants and prosthesis, such as stents, grafts, aneurysm coils, vascular filters, heart valves, active implants, cosmetic pouches or breast implants,
adhesives between tissue pieces, adhesives between tissues and devices, adhesives between artificial tissue and natural tissue, bioprobes,
pouches, patches, drug delivery media,
auxiliary devices, such as plasters, balloon expanding media,
or any combination thereof.

As noted above, the present invention is applicable to a system for the non-invasive application of heat to mammalian tissue and the methods related thereto. The present invention should not be considered limited to the particular examples described above, but rather should be understood to cover all aspects of the invention as fairly set out in the attached claims. Various modifications, equivalent processes, as well as numerous structures to which the present invention may be applicable will be readily apparent to those skilled in the art to which the present invention is directed upon review of the present specification. The claims are intended to cover such modifications and devices.

LEGEND to FIGS. 1 and 2

| | |
|---|---|
| 1 | medical device |
| 2 | inductor |
| 3 | alternating magnetic field (AMF) |
| 4 | resonant circuit or impedance matching network |
| 5 | alternating current power source |
| 6 | alternating current generator |
| 7 | catheter tube |
| 8 | expandable balloon |
| 9 | carotid artery |
| 12 | magnetic circuit |
| 13 | gap of magnetic circuit |
| 14 | patient |
| 15 | pole |

1. A system for applying heat to an area of the body of a mammal, comprising:
a) a device fabricated from or coated with a material comprising a non-metal matrix and susceptor particles;
b) an inductor and magnetic circuit for heating the particles by transmitting an alternating magnetic field (AMF); and
c) an alternating current generator providing an alternating current to the inductor.

2. The system according to claim 1, wherein the susceptor particles of the device possess a Curie temperature.

3. The system according to claim 2, wherein the susceptor particles comprise at least one of: a) SrFe₁₂O₁₉, Meₐ-2W, Meₐ-2Y, and Meₐ-2Z, wherein 2W is BaO:2 MeₐO:8Fe₂O₃, 2Y is 2(BaO: MeₐO: Fe₂O₃), and 2Z is 3BaO:2 MeₐO:12 Fe₂O₃, and wherein Meₐ is a divalent cation selected from Mg, Co, Mn and Zn; b) 1 Me_{b}O: 1 Fe₂O₃, where Me_{b}O is a transition metal oxide selected from Ni, Co, Mn, and Zn; c) La_{0.8}Sr_{0.2}MnO₃; d) Y₃Fe₅₋ₓMₓO₁₂ where M is Al, or Gd and 0<x<2; e) metal alloys of any combination of Pd, Co, Ni, Fe, Cu, Al, and Si; f) metal alloys of any combination of Gd, Tb, Dy, Ho, Er, and Tm with any combination of Ni, Co, and Fe; and g) metal alloys RMn₂X where R is a rare earth, such as La, Ce, Pr, or Nb and X is either Ge or Si.

4. The system according to claims 1-3, wherein the susceptor particles are coated with a polymeric material.

5. The system according to claims 1-4, wherein the matrix-particle mixture density is between 5% and 95% by volume.

6. The system according to claims 2-5, wherein the Curie temperature is from about 35° C. to about 150° C.

7. The system according to claims 2-5, wherein the Curie temperature is from about 37° C. to about 75° C.

8. The system according to claims 2-5, wherein the Curie temperature is from about 38° C. to about 45° C.

9. The system according to claims 1-8, wherein the particles are from about 10 nanometers to about 500 micrometer in the longest dimension.

10. The system according to claims 1-8, wherein the particles are from about 20 nanometers to about 200 nanometers in the longest dimension.

11. The system according to claims 1-8, wherein the particles are from about 1 micrometer to about 50 micrometers in the longest dimension.

12. The system according to claims 1-11, wherein the matrix material is a plastic, a thermoset, a thermoplastic, an elastomer, a ceramic, or a gel.

13. The system according to claim 12, wherein the gel is from a natural source, such as starch; is from an artificial source, such as polyacrylamide; is a sugar based, such as glactose; is wax based such as, esters of long-chain carboxylic acids with long-chain alcohols; is fat based, such as triesters of glycerol with three long-chain carboxylic acids; is from petrochemical oils or natural oils, such as coconut, corn, olive or bean oils; is selected from the group consisting of acrylonitriles, acrylic acids, polyacrylimides, acrylimides, acrylimidines, polyacrylonitriles, and polyvinylalcohols; is a hydrogel in its hydrated or dehydrated form; or is silicone based.

14. The system according to claim 12, wherein the matrix material is an absorbable or bioabsorbable material.

15. The system according to claims 1-14, wherein the device is implanted in the body for at least one hour and can be repeatedly heated.

16. The system according to claims 1-15, wherein only a portion of the device comprise a matrix with embedded susceptible particles.

17. The system according to claims 1-16, wherein the device is a surgical tool, a catheter, a tube, a balloon catheter, a balloon, the balloon expanding media, a guide wire, a stent, a graft, an aneurism coil, a vascular filter, a heart valve, a prosthesis of any kind, a plaster, a needle of any kind, a nail, a screw, a suture, a clip, a localizer, a filament, a fibre, an active implant, a trocar, an open or minimal invasive surgical tool, an interventional tool, a bioprobe, the adhesive between two tissue pieces, the adhesive between a tissue and another device, the adhesive between an artificial tissue and a natural tissue, a drug delivery medium, a pouch, a patch, an ablation device, or any combination thereof.

18. A system according to claims 1-16, wherein the inductor and magnetic circuit are non-invasive.

19. A method of applying heat to a mammalian body, comprising:
a. applying to a mammal tissue a device that is partially or completely fabricated from or coated with a non-metal matrix containing susceptor particles, and
b. applying an AMF to the device.

20. The method according to claim 19, wherein, the susceptor particles have a Curie temperature.

21. The method according to claims 19-20, wherein the AMF frequency is between 50 Hz and 55 Mz.

22. The method according to claims 19-20, wherein the AMF frequency is between 20 kHz and 1 MHz.

23. The method according to claims 19-20, wherein the AMF frequency is between 50 kHz and 500 kHz.

24. The method according to claims 19-23, wherein the susceptor particles are heated from body temperature to the desired temperature in less than or equal to 40 seconds.

25. The method according to claims 19-23, wherein the susceptor particles are heated from body temperature to the desired temperature in less than or equal to 10 seconds.

26. The method according to claims 19-25, wherein applying the AMF to the device is performed non-invasively to the patient.
